(19) United States
(12) Patent Application Publication
(10) Pub. No.: US 2014/0045754 A1
(43) Pub. Date: Feb. 13, 2014

(54) GLP-1 COMPOSITIONS

(71) Applicants: Joern Drustrup, Farum (DK); Kasper Huus, Gentofte (DK); Per Balschmidt, Hoersholm (DK)

(72) Inventors: Joern Drustrup, Farum (DK); Kasper Huus, Gentofte (DK); Per Balschmidt, Hoersholm (DK)

(21) Appl. No.: 13/980,147

(22) PCT Filed: Jan. 19, 2012
(86) PCT No.: PCT/EP2012/050784

§ 371 Date: Oct. 28, 2013

Related U.S. Application Data
(60) Provisional application No. 61/436,785, filed on Jan. 27, 2011.

(30) Foreign Application Priority Data
Jan. 19, 2011 (EP) 11151455.0

(51) Int. Cl. A61K 38/26

(52) U.S. Cl. CPC A61K 38/26
USPC 514/11.7

(57) ABSTRACT

The present invention relates to pharmaceutical compositions comprising a GLP-1 compound, a divalent metal and a polycationic compound. The invention is characterised in that the GLP-1:divalent metal molar ratio is 1:>2. The compositions of the invention are particularly useful in the treatment of diabetes.

### TECHNICAL FIELD

The present invention relates to the field of pharmaceutical compositions comprising glucagon-like peptide-1 (GLP-1) compounds and methods of making them.

### BACKGROUND

Glucagon-like peptide-1 (GLP-1) is a gut hormone secreted in the body by the intestinal L cells. The natural active forms of GLP-1 are GLP-1-(7-37) and GLP-1-(7-36)NH2. GLP-1 and its analogs are promising treatments of diabetes mellitus, thanks to their ability to increase insulin secretion from the pancreas, insulin-sensitivity in both alpha cells and beta cells, satiety, and to decrease glucagon secretion from the pancreas.

Like most pharmaceutically relevant proteins and peptides, GLP-1 compounds are poorly absorbed through biological membranes. Therefore, they are typically administered by the parenteral route, by subcutaneous injection. In addition, GLP-1 compounds are unstable due to susceptibility towards various water catalyzed reactions when formulated into an aqueous solution.

The natural GLP-1 has a short half life in the body, few minutes, because it is rapidly degraded by the enzyme dipeptidyl peptidase-4. To overcome this disadvantage, sustained release technologies are the subject of considerable research. One approach is to prepare a suspension of the active ingredient which upon administration is slowly dissolved and released to the blood stream. In this perspective, analogs of the natural GLP-1 are being developed. Liraglutide (Arg³⁴,Lys²⁶(N^{ε}-(γ-Glu(N^{α}-hexadecanoyl)))-GLP-1(7-37) or also named N^{ε26}-[(4S)-4-carboxy-4-(hexadecanoylamino)butanoyl]-[Arg³⁴]-GLP-1-(7-37)-peptide) is one of them. It is commercialized under the trademark Victoza® as a once daily injectable medication. In this formulation, liraglutide has a pharmacokinetic profile (PK) lasting 1 day upon subcutaneous administration. This is a major achievement but there is still a need to lower the frequency of injections for the patients. The development of a once weekly injection medication would be another major achievement.

Some pharmaceutical compositions of the prior art combine GLP-1 compounds with a basic polypeptide and a divalent metal ion, such as zinc (WO02/098348) into particles in order to control the drug release. However, the compositions of the prior art are still not satisfactory and there is still a need for a GLP-1 product with a reduced frequency of injections, with reduced associated side effects and with advantageous physical properties.

### SUMMARY

The invention relates to new GLP-1 compositions.

The invention is based on the recognition that compositions comprising a GLP-1 compound and a divalent molar ratio in a specific molar ratio present beneficial properties. Surprisingly, it has been found that a composition comprising more than 2 molecules of a divalent metal per molecule of GLP-1 is associated with a significant increase in the time of action of the GLP-1 compound in the body while maintaining low or minimizing the problems encountered with GLP-1 formulations.

The invention may also solve further problems that will be apparent from the disclosure of the exemplary embodiments.

In one aspect, the invention relates to a composition comprising a GLP-1 compound, a divalent metal and a polycationic compound, wherein the GLP-1:divalent metal molar ratio is 1:>2.

In another aspect, the invention relates to methods for the preparation of such compositions.

In another aspect, the invention relates to the use of such composition as a medicament.

In one aspect, the invention provides an improved sustained release GLP-1 composition, with an increased duration of action of the GLP-1 compound. Also or alternatively, in another aspect, the invention provides a sustained release GLP-1 composition with improved physical properties, such as physical stability, a smooth injection through fine needles, an easy re-suspension. Also or alternatively, in another aspect, the invention provides a sustained release GLP-1 composition with improved chemical properties, such as a higher concentration of active ingredient available to patients or/and an efficient incorporation of the components in the composition. Also or alternatively, in embodiments where the composition comprises particles, the invention provides a GLP-1 composition with a higher control of the particles size, a reduced release of free components from the particles. Also or alternatively, in another aspect, the invention provides a sustained release GLP-1 composition with improved side effects, such as a lower burst release, a lower tissue reaction especially at injection site, a lower histamine release.

In another aspect, the invention provides an improved method of making a GLP-1 composition. Also or alternatively, in another aspect, the invention provides a simple method, with no or limited external intervention, e.g. where a final pH adjustment is avoided. Also or alternatively, in another aspect, the invention provides a method applicable for sterile conditions.

In another aspect, the invention provides a treatment with a reduced frequency in injections for patients.

The invention may also solve further problems that will be apparent from the disclosure of the exemplary embodiments.

### BRIEF DESCRIPTION OF DRAWINGS

FIG. 1 shows the optimisation of liraglutide:zinc molar ratios at various pH-values and various.

FIG. 2 shows the optimisation of liraglutide:protamine molar ratios.

FIG. 3 shows the optimisation of the pH value in a composition with a molar liraglutide:zinc:protamine ratio of 1:2,2:0,14.

### DESCRIPTION

The invention relates to novel GLP-1 pharmaceutical compositions. The novel compositions of the invention can be used for the treatment of diabetes, such as type 2 diabetes. The compositions are useful as a treatment with a frequency of administration below once per day.

The compositions of the invention give a suitable sustained release PK profile upon subcutaneous administration, are of appropriate and controlled physical and chemical properties, such as particles size when applicable, are easily resuspendable upon storage, and are injectable through fine injection needles. They also allow the GLP-1 compound to be formulated at high concentrations, allowing a longer time of action. This specific GLP-1:divalent metal molar ratio in the composition also reduces undesired side effects.

The features of the invention will be better understood in the description that follows.

In one aspect, the invention relates to a composition comprising a GLP-1 compound, a divalent metal and a polycationic compound, wherein the GLP-1:divalent metal molar ratio is 1:>2.

Non-limiting examples of GLP-1 compound include a natural GLP-1, a GLP-1 analogue or a GLP-1 derivative. In its broadest sense, the term “natural GLP-1” refers to a naturally occurring molecule of the glucagon family of peptides or of the family of exendins. The glucagon family of peptides are encoded by the pre-proglucagon gene and encompasses three small peptides with a high degree of homology, i.e. glucagon (1-29), GLP-1 (1-37) and GLP-2 (1-33). The term “natural GLP-1” also refers to the human GLP-1(7-37), the sequence of which is disclosed as SEQ ID NO:1 in WO 2006097537 and included herein by reference, and to the human GLP-1(7-36)NH2. Exendins are peptides expressed in lizards and like GLP-1, are insulinotropic. Examples of naturally occurring exendins are exendin-3 and exendin-4.

In a particular embodiment, the term “natural GLP-1” refers to glucagon (1-29), GLP-1 (1-37) and GLP-2 (1-33), the human GLP-1(7-37)), the human GLP-1(7-36)NH2, exendin-3 and exendin-4.

In a particular embodiment, the term “GLP-1 compound” does not include the human GLP-1(7-36)NH2. In a particular embodiment, the term “GLP-1 compound” does not include the human GLP-1(7-37).

In a particular embodiment, the term “GLP-1 compound” does not include glucagon.

In a particular embodiment, the term “GLP-1 compound” does not include the human GLP-1(7-36)NH2 and glucagon or does not include human GLP-1(7-36)NH2, human GLP-1(7-37) and glucagon.

In a more particular embodiment, the term “natural GLP-1” only refers to the human GLP-1(7-37).

The term “analogue” as used herein referring to a peptide means a modified peptide wherein one or more amino acid residues of the peptide have been substituted by other amino acid residues and/or wherein one or more amino acid residues have been deleted from the peptide and/or wherein one or more amino acid residues have been added to the peptide. Such addition or deletion of amino acid residues can take place at the N-terminal of the peptide and/or at the C-terminal of the peptide.

In its broadest sense, the term “GLP-1 analogue” or “analogue of GLP-1” as used herein refers to an analogue of a natural GLP-1. It does not include a natural GLP-1 as such as defined herein. In particular, the term “GLP-1 analogue” does not include glucagon (1-29), GLP-1 (1-37) and GLP-2 (1-33), the human GLP-1(7-37)), the human GLP-1(7-36)NH2, exendin-3 and exendin-4.

In a particular embodiment, the term “GLP-1 analogue” or “analogue of GLP-1” as used herein refers to an analogue of human GLP-1(7-37) or GLP-1(7-36)NH2.

Non-limiting examples of GLP-1 analogues comprise exenatide and taspoglutide.

In a particular embodiment, the “GLP-1 analogues” comprise analogues with a maximum of 17 amino acid modifications (i.e. up to 17 amino acids have been modified in total, where the changes can be amino acid substitutions, additions and/or deletions) compared to a natural GLP-1 of reference or, in particular, compared to human GLP-1-(7-36)NH2 or GLP-1(7-37).

All amino acids for which the optical isomer is not stated is to be understood to mean the L-isomer.

In embodiments of the invention a maximum of 17 amino acids have been modified (substituted, deleted, added or any combination thereof) relative to a natural GLP-1 of reference or, in particular, relative to human GLP-1-(7-36)NH2 or GLP-1(7-37). In embodiments of the invention a maximum of 15 amino acids have been modified. In embodiments of the invention a maximum of 10 amino acids have been modified. In embodiments of the invention a maximum of 8 amino acids have been modified. In embodiments of the invention a maximum of 7 amino acids have been modified. In embodiments of the invention a maximum of 6 amino acids have been modified. In embodiments of the invention a maximum of 5 amino acids have been modified. In embodiments of the invention a maximum of 4 amino acids have been modified. In embodiments of the invention a maximum of 3 amino acids have been modified. In embodiments of the invention a maximum of 2 amino acids have been modified. In embodiments of the invention 1 amino acid has been modified relative to a natural GLP-1 of reference or, in particular, relative to human GLP-1-(7-36)NH2 or GLP-1(7-37). In a particular embodiment, the amino acid modifications of this paragraph are relative to human GLP-1(7-37).

In a particular embodiment, the GLP-1 analogues comprise a substitution of the amino acid residue in position 34 from Lys to Arg, i.e. Arg³⁴, compared to GLP-1(7-37) or GLP-1-(7-36)NH2. In a particular embodiment, the GLP-1 analogues have a substitution of the amino acid residue in position 8 from Ala to Aib (alpha-amino-iso-butyric acid), i.e. Aib⁸. In a particular embodiment, the GLP-1 analogues have the Arg³⁴ substitution, the Aib⁸ substitution, or both the Arg³⁴ and Aib⁸ substitutions, and possibly one more amino acid modification compared to GLP-1(7-37) or GLP-1-(7-36)NH2. In a particular embodiment, the amino acid modifications of this paragraph are relative to human GLP-1(7-37).

The term “derivative” as used herein in relation to a peptide means a chemically modified peptide or an analogue thereof, wherein at least one substituent has been attached to the unmodified peptide or an analogue thereof, i.e. a peptide which has been covalently modified. The substituent may also be referred to as a “side chain”. The peptide to which the substituent(s) is attached may also be referred to as the “parent” peptide.

In its broadest sense, the term “GLP-1 derivative” or “derivative of GLP-1” as used herein refers to a derivative of a parent peptide selected from a natural GLP-1 or an analogue thereof. It does not include a natural GLP-1 as such as defined herein. In particular, the term “GLP-1 derivative” does not include glucagon (1-29), GLP-1 (1-37) and GLP-2 (1-33), the human GLP-1(7-37)), the human GLP-1(7-36)NH2, exendin-3 and exendin-4.

In a particular embodiment, the term “GLP-1 derivative” or “derivative of GLP-1” refers to a derivative of a parent peptide selected from human GLP-1(7-37) or GLP-1(7-36)NH2 or an analogue thereof.

In a particular embodiment, the term “GLP-1 derivative” or “derivative of GLP-1” as used herein refers to a derivative of a parent peptide selected from a GLP-1 analogue, where said analogue comprises a maximum of 17 amino acid modifications compared to a natural GLP-1 of reference or, in particular, compared to human GLP-1-(7-36)NH2 or GLP-1(7-37), or, in particular, compared to human GLP-1(7-37). In one embodiment, the “GLP-1 derivative”, in particular when defined in comparison to GLP-1(7-37), does not include GLP-1(7-36)NH2.

Typical modifications are amides, carbohydrates, alkyl groups, acyl groups, esters, polyethylene glycol (PEG) groups, sialylation groups, glycosylation groups and the like of a parent peptide. In one embodiment, the parent peptide is a GLP-1 analogue as defined above.

In particular embodiments, the side chain has at least 10 carbon atoms, or at least 15, 20, 25, 30, 35, or at least 40 carbon atoms. In further particular embodiments, the side chain may further include at least 5 hetero atoms, in particular O and N, for example at least 7, 9, 10, 12, 15, 17, or at least 20 hetero atoms, such as at least 1, 2, or 3 N-atoms, and/or at least 3, 6, 9, 12, or 15 O-atoms.

In one embodiment, the term “GLP-1 derivative” refers to acylated GLP-1 parent peptide. In a particular embodiment, the term “GLP-1 derivative” refers to acylated GLP-1 parent peptide where the parent peptide is selected from a GLP-1 analogue comprising a maximum of 17 amino acid modifications compared to a natural GLP-1 of reference or, in particular, compared to human GLP-1-(7-36)NH2 or GLP-1(7-37).

The side chain may be covalently attached to a lysine residue of the GLP-1 parent peptide by acylation. Additional or alternative conjugation chemistry includes alkylation, ester formation, or amide formation, or coupling to a cysteine residue, such as by maleimide or haloacetamide (such as bromo-/fluoro-/iodo-) coupling.

For the preparation, an active ester of the side chain is covalently linked to an amino group of a lysine residue, preferably the epsilon amino group thereof, under formation of an amide bond (this process being referred to as acylation).

Preferred side chains include, for example, fatty acids and fatty diacids. The term fatty acid refers to aliphatic monocarboxylic acids having from 4 to 28 carbon atoms. The fatty acid may be branched or unbranched. The fatty acid is preferably even numbered. The fatty acid may be saturated or unsaturated. The term fatty diacid refers to fatty acids as defined above but with an additional carboxylic acid group in the omega position. Thus, fatty diacids are dicarboxylic acids.

In a particular embodiment, the side chain(s) is a fatty acid having 10 to 20 carbon atoms, and preferably 14 to 20 or 16 to 18 carbon atoms, optionally with a spacer.

In a particular embodiment, the side chain(s) is a fatty acid of formula Chem.1: HOOC(CH₂)ₘCO, wherein m is an integer from 8 to 18, optionally with a linker. In a particular embodiment, m is an integer from 12 to 18 or from 14 to 16.

In a particular embodiment, the side chain(s) is selected from the group consisting of HOOC(CH₂)₁₄CO—, HOOC(CH₂)₁₆CO—, HOOC(CH₂)₂₂CO—, CH₃(CH₂)₁₄CO—, CH₃(CH₂)₁₆CO— and CH₃(CH₂)₁₈CO—.

In one embodiment, the term “GLP-1 derivative” comprises or refers to monoacylated GLP-1 parent peptide, i.e. a GLP-1 parent peptide comprising only one acylation as defined above.

In a particular embodiment, the side chain is a fatty acid or a fatty diacid of which an acid group forms an amide bond with the epsilon amino group of a lysine residue in the GLP-1 compound, preferably via a spacer. In one embodiment, said lysine residue is Lys²⁶, especially when the parent peptide is human GLP-1(7-37), GLP-1(7-36)NH2 or a GLP-1 analogue.

In a particular embodiment, the side chain is attached to the parent peptide by means of a linker. In a particular embodiment, the linker comprises a γ-glutamic acid (γ-Glu) and/or 1, 2 or 3 OEG molecules. In γGlu the gamma carboxy group of the amino acid glutamic acid is used for connection to another linker element, or to the epsilon-amino group of lysine. An OEG molecule is also named a di-radical of 8-amino-3,6-dioxaoctanic acid, and/or it may be represented by the formula Chem. 2: —NH—(CH2)2-O—(CH2)2-O—CH2-CO—.

The linker may include one or more γGlu, and/or one or more OEG. More in particular, the γGlu and OEG linker elements may, independently, be used p times where p is zero or an integer in the range of 1-3. Examples of preferred linkers are γGlu, γGlu-2×OEG, and γGlu-3×OEG where in all cases the alpha-amino group of Glu forms an amide bond with the carboxy group of the protracting moiety.

In a particular embodiment, the GLP-1 derivative is a derivative of a GLP-1 analogue which comprises the Arg³⁴ substitution or the Arg³⁴ and the Aib⁸ substitutions compared to human GLP-1(7-37), GLP-1(7-36)NH2 and which comprises a side chain attached to Lys²⁶. In a particular embodiment said side chain is a fatty acid as defined above, especially a fatty acid of formula Chem.1, with m being an integer from 8 to 18, optionally with a linker being γGlu.

In one embodiment, the GLP-1 derivative is as defined in the patent applications WO 98/08871 and WO 06/097537, entirely included herein by reference. Non-limiting examples of monoacylated GLP-1 derivatives can be found in those applications.

Non-limiting examples of GLP-1 derivatives also include:
N^{ε37}-[2-[2-[2-[[2-[2-[2-[[(4S)-4-carboxy-4-[[4-[(19-carboxynonadecanoylamino)methyl]cyclo hexanecarbonyl]amino]butanoyl]amino]ethoxy]ethoxy]acetyl]amino]ethoxy]ethoxy]acetyl]-[Imp⁷,Glu²²,Arg²⁶,Arg³⁴,Lys³⁷]-GLP-1-(7-37)-peptide;
N^{ε26}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[Aib⁸,Arg³⁴]-GLP-1-(7-37)-peptide, also called semaglutide;
N^{ε26}-[(4S)-4-carboxy-4-(hexadecanoylamino)butanoyl]-[Arg³⁴]-GLP-1-(7-37)-peptide, also called liraglutide;
N^{ε26}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl],N^{ε37}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl]-[Aib⁸,Arg³⁴,Lys³⁷]-GLP-1-(7-37)-peptide;
N^{ε26}-[2-[2-[2-[[2-[2-[2-[[(4S)-4-carboxy-4-[12-(3-carboxyphenoxy)dodecanoylamino]butanoyl]amino]ethoxy]ethoxy]acetyl]amino]ethoxy]ethoxy]acetyl],N^{ε37}-[2-[2-[2-[[2-[2-[2-[[(4S)-4-carboxy-4-[12-(3-carboxyphenoxy)dodecanoylamino]butanoyl]amino]ethoxy]ethoxy]acetyl]amino]ethoxy]ethoxy]acetyl]-[Aib⁸,Arg³⁴,Lys³⁷]-GLP-1-(7-37)-peptide;
lixisenatide;
albiglutide;
dulaglutide.

In a particular embodiment, the GLP-1 derivative is liraglutide or is semaglutide. The chemically modified derivatives of natural GLP-1 can be prepared for example as described in U.S. Pat. No. 6,451,762 or in Knudsen et. al. (2000) J Med Chem 43, 1664-1669.

Non-limiting examples of divalent metal include zinc (Zn), calcium (Ca), manganese (Mn) or magnesium (Mg). As non-limiting examples, the source of zinc may be zinc chloride, zinc acetate, zinc sulphate or zinc oxide. Amongst these, at least, zinc acetate allows an easy preparation of solutions. The divalent metal stabilizes the composition during storage. It helps minimizing the burst release and associated side effects.

Non-limiting examples of polycationic compound include protamine, chitosan, a chitosan derivative, polylysine or polyarginine. As non-limiting examples, protamine can come from protamine chloride, protamine acetate, protamine sulphate. The polycationic compound helps controlling the physical properties of the composition. It also helps improving the sustained release.

The various features of the composition contribute in the optimisation of the composition properties and advantages.

In one embodiment, the GLP-1:divalent metal molar ratio in the composition is 1:>2. This means that the composition comprises more than 2 divalent metal molecules per GLP-1 molecule. In another embodiment, the GLP-1:divalent metal molar ratio in the composition is 1:≧2,1 or of 1:>2,1 or 1:2,1. In another embodiment, the GLP-1:divalent metal molar ratio in the composition is 1:≧2,2 or of 1:>2,2 or 1:2,2. In another embodiment, the GLP-1:divalent metal molar ratio is between 1:2,0 and 1:2,4, between 1:2,1 and 1:2,4 or between 1:2,1 and 1:2,3. These embodiments avoid excess of divalent metal molecules. They also advantageously limit the presence of free GLP-1 and of free divalent metal molecules in the supernatant, especially when the composition is in the form of particles or a suspension of particles. Free divalent metal molecules may otherwise generate unwanted tissue reaction.

The above ratios are associated with a reduction or with a substantial reduction of the burst release and related side effects such as injection site reaction. It also increases the chemical and physical stability of the composition and of the GLP-1 molecule itself. It also helps controlling and increasing the sustained-release of the GLP-1 compound after injection into the body and the associated protraction action.

In one embodiment, the GLP-1:polycationic compound molar ratio in the composition is 1:>0,01. In another embodiment, the GLP-1:polycationic compound molar ratio is 1:0,01-1; 1:>0,10; 1:>0,11; 1:≧0,11; 1:≧0,12; 1:>0,12; 1:0,12-0,15; 1:≧0,13; 1:>0,13; 1:0,13-0,15; 1:1,13; 1:0,14-1:0,15; 1:0,14 or 1:0,15. These embodiments advantageously limit the presence of free polycationic compound in the supernatant, especially when the composition is in the form of particles or of a suspension of particles.

In one embodiment, the composition of the invention comprises a GLP-1 compound concentration of up to 100 mg/mL or between 0,1 and 100 mg/mL. In one embodiment, the composition of the invention comprises a GLP-1 compound concentration between 35 and 45 mg/mL, between 37 and 43 mg/mL or of 40 mg/mL. For example, a composition comprising up to 40 mg/mL of liraglutide has been obtained in the final suspension. These concentrations not only but especially concern a final composition, ready for injection.

In one embodiment, the composition is an aqueous composition.

In one embodiment, the composition is in the form of particles, not yet in suspension.

In one embodiment, the pharmaceutical composition of the invention is in the form of a suspension of particles.

In one embodiment, it is a suspension of particles into an aqueous vehicle.

As a further advantage, the GLP-1 compounds are stabilized against chemical and physical degradation in the composition of the invention.

In one embodiment, the pharmaceutical composition of the invention is in the form of a non-aqueous suspension of particles. The non-aqueous medium can be, as a non-limiting example, an oil, such as MCT (medium chain triglyceride). The composition can be in a form that is ready-to-use, for examples where particles are pre-mixed into a suspension, or the composition can be stored in a form that needs to be mixed before use, i.e. in the form of particles only, not yet in suspension.

In another embodiment, the pharmaceutical composition of the invention is in the form of a suspension of particles wherein the particles can be further incorporated into at least one biodegradable polymer, such as PLGA (poly(lactic-co-glycolic acid), the resulting combination being either present as spheres or rods. The spheres, consisting of both the particles and at least one biodegradable polymer, can either be premixed into an oil such as MCT, and thereby ready to use, or separately stored i.e. in the form of spheres only, not yet in suspension. In the latter case, mixing of the spheres and medium has to take place before use. In addition, the obtained spheres can be stored separately from an aqueous medium. In this case, mixing of the spheres and aqueous medium has to take place shortly before use, in order to avoid the biodegradable polymer to degrade prior to dosing

In another embodiment, the pharmaceutical composition of the invention may also comprise one or several of the followings:

a tonifier or isotonic agent, such as sodium chloride, glycerol, propylene glycol, mannitol, sucrose, trehalose;

a buffer, such as TRIS (tris(hydroxymethyl)aminomethane), HEPES (4-(2-hydroxyethyl)-1-piperazineethanesulfonic acid), GlyGly etc, possibly with a pH adjusting agent such as hydrochloric acid, sodium hydroxide, acetic acid etc;

a preservative agent, such as phenol, m-cresol, benzyl alcohol etc, and mixtures thereof;

an additional stabilizer, such as amino acids, surfactants etc.

These additional components are especially appropriate with aqueous compositions.

In one embodiment, the composition of the invention has a pH between 4 and 8,2. In another embodiment, said pH is between 7,2 and 8,2, between 7,4 and 8,2, between 7,4 and 7,9, between 7,6 and 8,0 or between 7,7 and 7,9, or said pH is 7,4, 7,6, 7,8, 8,0 or 8,2. If not specified otherwise, a pH value is considered at around room temperature, e.g. 20-26° C. or 23-25° C.

Thus, it is possible to achieve a composition easy to inject through fine needles, with low side effects and with a suitable sustained release profile after administration.

In another embodiment, the Zn:GLP-1 molar ratio does not exceed a value where not all zinc is efficiently incorporated into the composition, especially at high pH value, in order to avoid the formation of zinc hydroxide (Zn(OH)₂) precipitates. These zinc hydroxide precipitates may cause serious tissue reaction at the injection site.

The invention is particularly useful as an injectable, for example, without limitation, for subcutaneous, intramuscular or intraperitoneal administration route.

In one embodiment, the composition of the invention allow a time release of the GLP-1 compound after injection into the body (in vivo plasma profile) of more than 24 hours, of more than 72 hours, of up to 3 days, 4 days, 5 days, 7 days or of up to 8 days.

In another embodiment, the compositions of the invention allow a time release of the GLP-1 compound after injection into the body (in vivo plasma profile) of more than 7 days, more than 8 days, more than 9 days, more than 10 days, more than 14 days, more than 15 days, more than 20 days, more than 21 days, more than 29 days, more than 30 days, more than 31 days, or of up to 1 month.

In particular, non-aqueous compositions of the invention or compositions comprising a biodegradable polymer, or both can provide an even further sustained release profile for the GLP-1 molecule, as compared to the release profile of GLP-1 from merely aqueous compositions.

In one embodiment, the composition of the invention presents low burst release when injected into a target body.

In one embodiment, the composition of the invention, when in the form of a suspension, presents a low sedimentation rate. For example, the composition of the invention can have a sedimentation percentage higher than 80% at 5 minutes after being resuspended, such as 90 or 95%. A sedimentation percentage higher than 80% at 5 minutes means that the solids in this suspension settle less than 20% (of the total height of the suspension) within these 5 minutes.

In one embodiment, the composition of the invention is readily injectable through an injection needle finer than or equal to a 28G (gauge) or a 30G regular walled needle, for example with a dose force, i.e. injection force, lower than 25N (newtons) at a dosing rate of at least 50 μL/sec.

In one embodiment, the composition of the invention is in the form of a suspension of particles. Indeed, the GLP-1 compound, the divalent metal and the polycationic compound can be aggregated together and form particles.

The term “particles” as used herein means a solid material complex. In one embodiment, the particles comprise the GLP-1 compound, the divalent metal and the polycationic compound. In one embodiment, the particles comprise a core and a surrounding layer. In one embodiment, the core comprises the GLP-1 compound and the divalent metal, and the layer comprises the polycationic compound, this layer being present on the surface of the core, surrounding the core. The layer coats the surface of the core and is a part of the particle. The polycationic compound forming the surrounding layer is attached onto the surface of the core of the particle. This contributes to limit the presence of free polycationic compound in the supernatant. In particular, the term “layer” does not designate a composition in which the core would simply be suspended.

In another embodiment, the core of the particles consists of a GLP-1 compound and a divalent metal. In another embodiment, the core of the particles comprises no protamine. This reduces the formation of a gel consistency associated with the mixing of GLP-1 and protamine.

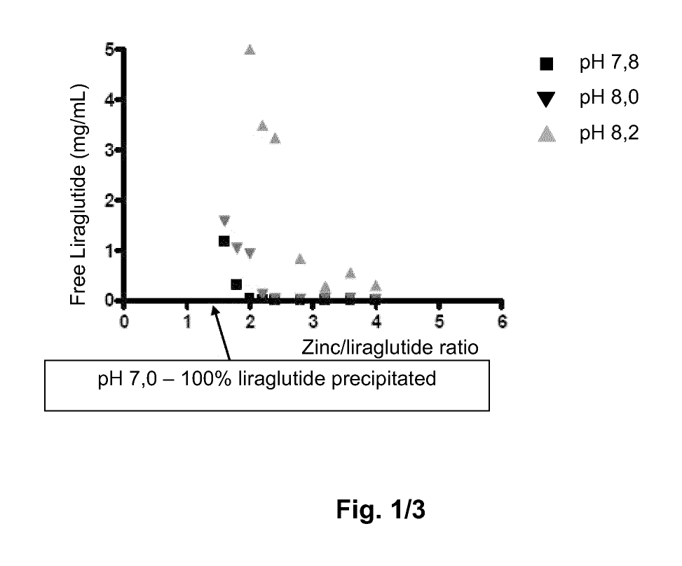

In one embodiment, the GLP-1 and the metal molecules are co-precipitated and form a homogenous mixture, which can be in the form of an amorphous complex or in the form of a crystalline complex. The term “homogenous” means that each component is evenly distributed in the mixture. The divalent metal reduces the release of free GLP-1 out of the particles into the supernatant, i.e. into the composition comprising the particles, before it is injected. (See FIG. 1 and example 1) This helps minimizing the burst release and related side effects such as injection site reaction. The polycationic compound helps reducing the injection site reaction and increases the sustained release.

In one embodiment, the polycationic compounds form a layer around the GLP-1 and divalent metal mixture. In one embodiment, this layer covers the major part of the surface of the mixture, so that only a minor part of the surface of the mixture is in direct contact with the external environment. In another embodiment, this layer covers the entire outer surface of the mixture, so that no component of the mixture is in direct contact with the external environment. In one embodiment, the mixture and the layer form a particle and the surrounding layer is the outer layer of the particle. It improves an efficient incorporation of the divalent metal and the polycationic compound in the composition and reduces the histological response at injection site. In one embodiment, the particles have a volumetric diameter below 200 μm. The term “diameter” as used herein designates the diameter of an entire particle. In the context of a composition comprising the particles of the invention, the “diameter” designates the mean diameter of either all or a proportion of the particles. In one embodiment, at least, 50% of the particles of the invention have a volumetric diameter less than 60 μm. In one embodiment, 50% of the particles of the invention have a volumetric diameter less than 40 μm. In one embodiment, 50% of the particles of the invention have a volumetric diameter in the range of 5-35 μm. The particle size distributions including the volumetric diameters can be determined using a Helos particle analyser from Sympatec, that uses a laser diffraction sensor.

By isophane titration studies, it has been possible to determine the required minimum or optimum concentration of divalent metal, as well as of polycationic compound, in order to obtain the most efficient incorporation of all three components in the composition, which is reflected by the amount of each components present in a free form in the supernatant, which is one aspect of the composition stability.

As shown in FIG. 1, the pH influences the molar ratios of the components allowing an optimum composition stability and, subsequently, optimum beneficial properties. For example, at room temperature and pH 7, a minimum of 1,3 zinc per GLP-1 molecule is needed to have no GLP-1 present in a free form in the composition, and at room temperature and pH 7,8, more than 2 zinc per GLP-1 molecule is needed to completely prevent the release of free GLP-1 into the supernatant. A GLP-1:zinc molar ratio between 1:2,1 and 1:2,2, or of 1:2,1 or 1:2,2 is used to compensate the pH deviation that may occur in a pharmaceutical formulation, ensuring that no free GLP-1 is released into the supernatant at any time during the formulation storage.

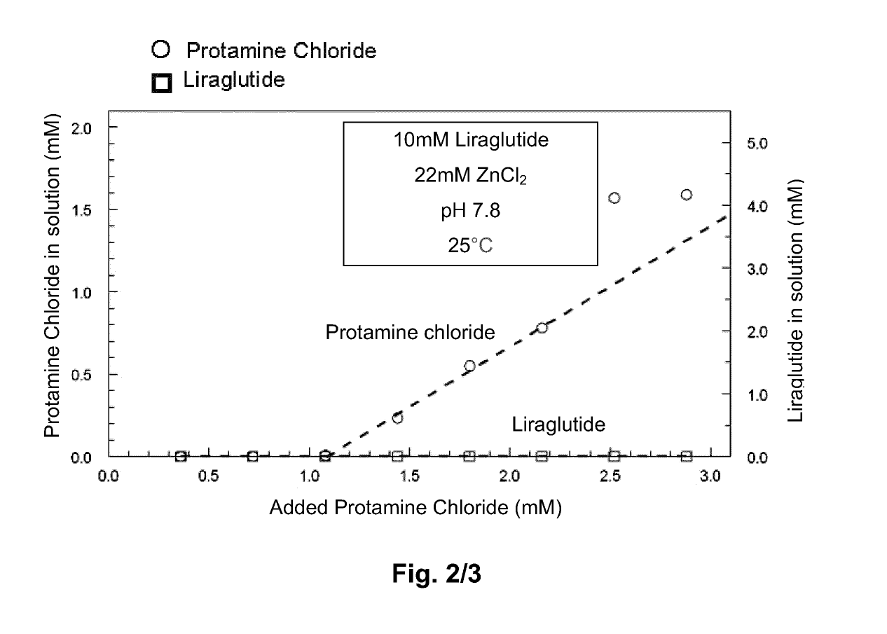

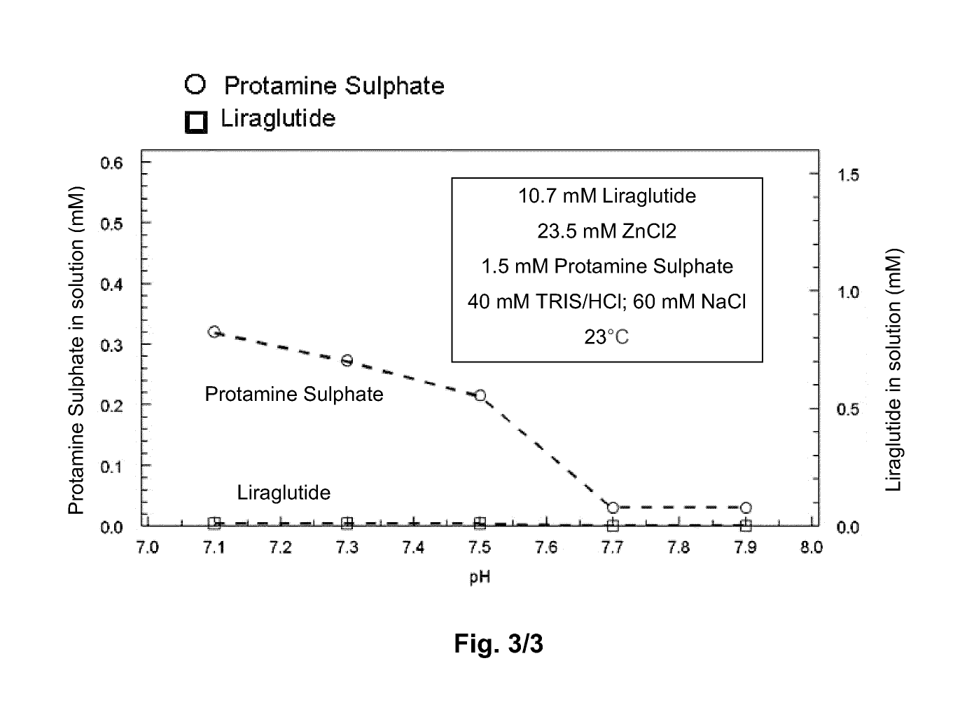

As shown in FIG. 2, the molar ratio between the polycationic compound and the GLP-1 compound influences the composition stability. As shown in FIG. 3, the pH also impacts. For example, at pH 7,8, with 0,11 molecule of protamine per molecule of liraglutide a good composition stability is achieved as well as an efficient incorporation of protamine in the composition. A molar ratio of 0,13, 0,14 or 0,15 polycationic compound per 1 GLP-1 compound compensates the pH variations in the composition.

The following embodiments are also part of the present invention:

A composition comprising a GLP-1 compound, a divalent metal and a polycationic compound wherein:
1—the GLP-1:divalent metal molar ratio is 1:>2.
2—the GLP-1 compound is selected from the group consisting of GLP-1 analogues and GLP-1 derivatives and the GLP-1:divalent metal molar ratio is 1:>2.
3—the GLP-1 compound is selected from the group consisting of GLP-1 analogues and GLP-1 derivatives and the GLP-1:divalent metal molar ratio is 1:2,1-2,4.
4—the GLP-1 compound is selected from the group consisting of GLP-1 analogues and GLP-1 derivatives and the pH is between 7,4 and 8,0 or 7,7 and 8,0.
5—the GLP-1 compound is selected from the group consisting of GLP-1 analogues and GLP-1 derivatives, the GLP-1:divalent metal molar ratio is 1:2,1-2,4 and the pH is between 7,4 and 8,0 or between 7,4 and 7,9.
6—the GLP-1 compound is selected from the group consisting of GLP-1 analogues and GLP-1 derivatives, the GLP-1:divalent metal molar ratio is 1:2,1-2,4 and the pH is between 7,4 and 8,0 or between 7,4 and 7,9, the GLP-1 compound, the divalent metal and the polycationic compound together form particles.
7—the GLP-1 compound is selected from the group consisting of GLP-1 analogues and GLP-1 derivatives, the GLP-1:divalent metal molar ratio is 1:2,1-2,4 and the pH is between 7,4 and 8,0 or between 7,4 and 7,9, the GLP-1 compound, the divalent metal and the polycationic compound form particles, the particles comprising a core and a surrounding layer, the core comprising the GLP-1 compound and the divalent metal and the layer comprising the polycationic compound.
8—the GLP-1 compound is selected from the group consisting of GLP-1 analogues and GLP-1 derivatives, the GLP-1:divalent metal molar ratio is 1:2,1-2,4 and the pH is between 7,7 and 8,0 or between 7,7 and 7,9.
9—the GLP-1:divalent metal molar ratio is 1:>2 and the GLP-1:polycationic compound molar ratio is 1:0,01-1.
10—the GLP-1:divalent metal molar ratio is 1:2,1-2,4 and the GLP-1:polycationic compound molar ratio is 1:0,13-0,15.
11—the GLP-1 compound is selected from the group consisting of GLP-1 analogues and GLP-1 derivatives, the GLP-1:divalent metal molar ratio is 1:2,1-2,4 and the GLP-1:polycationic compound molar ratio is 1:0,13-0,15.
12—the GLP-1 compound is selected from the group consisting of GLP-1 analogues and GLP-1 derivatives, the GLP-1:divalent metal molar ratio is 1:2,1-2,4, the GLP-1:polycationic compound molar ratio is 1:0,13-0,15 and the GLP-1 compound, the divalent metal and the polycationic compound form particles.
13—the GLP-1 compound is selected from the group consisting of GLP-1 analogues and GLP-1 derivatives, the GLP-1:divalent metal molar ratio is 1:2,1-2,4, the GLP-1:polycationic compound molar ratio is 1:0,13-0,15, the GLP-1 compound, the divalent metal and the polycationic compound form particles, the particles comprising a core and a surrounding layer, the core comprising the GLP-1 compound and the divalent metal and the layer comprising the polycationic compound.
14—the GLP-1 compound is selected from the group consisting of GLP-1 analogues and GLP-1 derivatives, the GLP-1:divalent metal molar ratio is 1:2,1-2,4, the GLP-1:polycationic compound molar ratio is 1:0,13-0,15 and the GLP-1 concentration is between 35 and 45 mg/ml or is 40 mg/ml in the composition.
15—the GLP-1 compound is selected from the group consisting of GLP-1 analogues and GLP-1 derivatives and the pH is between 7,4 and 8,0 or 7,7 and 8,0 and the GLP-1:polycationic compound molar ratio is 1:0,13-0,15.
16—the GLP-1 compound is selected from the group consisting of GLP-1 analogues and GLP-1 derivatives, the GLP-1:divalent metal molar ratio is 1:2,1-2,4 and the pH is between 7,4 and 8,0 or between 7,4 and 7,9 and the GLP-1:polycationic compound molar ratio is 1:0,13-0,15.
17—the GLP-1 compound is selected from the group consisting of GLP-1 analogues and GLP-1 derivatives, the GLP-1:divalent metal molar ratio is 1:2,1-2,4, the pH is between 7,4 and 8,0 or between 7,4 and 7,9, the GLP-1:polycationic compound molar ratio is 1:0,13-0,15 and the GLP-1 concentration is between 35 and 45 mg/ml or is 40 mg/ml in the composition.
18—the GLP-1 compound is selected from the group consisting of GLP-1 analogues and GLP-1 derivatives, the GLP-1:divalent metal molar ratio is 1:2,1-2,4 and the pH is between 7,7 and 8,0 or between 7,7 and 7,9 and the GLP-1:polycationic compound molar ratio is 1:0,13-0,15.
19—the GLP-1 compound does not include human GLP-1(7-36)NH2 and glucagon or does not include human GLP-1(7-36)NH2, human GLP-1(7-37) and glucagon.
20—the GLP-1 compound is a GLP-1 analogue or a GLP-1 derivative wherein the GLP-1 analogue is selected from the group consisting of analogues of human GLP-1(7-37) or GLP-1(7-36)NH2 with a maximum of 17 amino acid modifications compared to human GLP-1-(7-36)NH2 or GLP-1(7-37) and the GLP-1 derivative is selected from the group consisting of a derivative of human GLP-1(7-37), of GLP-1(7-36)NH2 or of an analogue thereof with a maximum of 17 amino acid modifications.
21—the GLP-1 compound is a GLP-1 derivative.
22—the GLP-1 compound is a GLP-1 derivative of human GLP-1(7-37), of GLP-1(7-36)NH2 or of an analogue thereof with a maximum of 17 amino acid modifications.
23—the GLP-1 compound is a GLP-1 derivative selected from the group consisting of amidated parent peptide, alkylated parent peptide, acylated parent peptide, esterified parent peptide, PEGylated parent peptide and/or sialylated parent peptide and the parent peptide is human GLP-1(7-37), GLP-1(7-36)NH2 or an analogue thereof with a maximum of 17 amino acid modifications.
24—the GLP-1 compound is a GLP-1 derivative selected from the group consisting of acylated GLP-1 parent peptide, the parent peptide is human GLP-1(7-37), GLP-1(7-36)NH2 or an analogue thereof with a maximum of 17 amino acid modifications, the parent peptide is acylated with a lipophilic substituent selected from the group consisting of aliphatic monocarboxylic or dicarboxylic acids having from 4 to 28 carbon atoms.
25—the GLP-1 compound is a GLP-1 derivative selected from the group consisting of acylated GLP-1 parent peptide, the parent peptide is human GLP-1(7-37), GLP-1(7-36)NH2 or an analogue thereof with a maximum of 17 amino acid modifications, the parent peptide is acylated with a lipophilic substituent selected from the group consisting of aliphatic monocarboxylic or dicarboxylic acids having from 14 to 20 carbon atoms.
26—the GLP-1 compound is selected from the group consisting of liraglutide, semaglutide, taspoglutide, exenatide, lixisenatide, albiglutide, dulaglutide, or N^{ε37}-[2-[2-[2-[[2-[2-[2-[[(4S)-4-carboxy-4-[[4-[(19-carboxynonadecanoylamino)methyl]cyclo hexanecarbonyl]amino]butanoyl]amino]ethoxy]ethoxy]acetyl]amino]ethoxy]ethoxy]acetyl]-[Imp⁷,Glu²²,Arg²⁶,Arg³⁴,Lys³⁷]-GLP-1-(7-37)-peptide or N^{ε26}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl],N^{ε37}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl]-[Aib⁸,Arg³⁴,Lys³⁷]-GLP-1-(7-37)-peptide or N^{ε26}-[2-[2-[2-[[2-[2-[2-[[(4S)-4-carboxy-4-[12-(3-carboxyphenoxy)dodecanoylamino]butanoyl]amino]ethoxy]ethoxy]acetyl]amino]ethoxy]ethoxy]acetyl],N^{ε37}-[2-[2-[2-[[2-[2-[2-[[(4S)-4-carboxy-4-[12-(3-carboxyphenoxy)dodecanoylamino]butanoyl]amino]ethoxy]ethoxy]acetyl]amino]ethoxy]ethoxy]acetyl]-[Aib⁸,Arg³⁴,Lys³⁷]-GLP-1-(7-37)-peptide.
27—any of embodiments 1 to 26 wherein the divalent metal is zinc and the polycationic compound is protamine.
For clarity reasons, the specific combinations of any one of embodiments 1 to 18 with any one of embodiments 19 to 26 are not reported here in writing but should be considered to be part of the disclosure.
Similarly, the specific combination of embodiment 27 with any one of embodiments 1 to 18 and with any one of embodiments 19 to 26 are not reported here in writing but should be considered to be part of the disclosure.
28—the GLP-1 compound is liraglutide, the divalent metal is zinc, and the liraglutide:zinc molar ratio is 1:2,1-2,4
29—the GLP-1 compound is liraglutide, the divalent metal is zinc, and the liraglutide:zinc molar ratio is 1:>2 and liraglutide concentration is between 35 and 45 mg/ml or is 40 mg/ml in the composition;
30—the GLP-1 compound is liraglutide, the divalent metal is zinc, and the liraglutide:zinc molar ratio is 1:2,1-2,4 and liraglutide concentration is between 35 and 45 mg/ml or is 40 mg/ml in the composition;
31—the GLP-1 compound is liraglutide, the divalent metal is zinc, and the liraglutide:zinc molar ratio is 1:2,1-2,4 or is 1:2,2, the composition having a pH≧7,7, a pH≧7,8, a pH between 7,7 and 8,0, a pH between 7,7 and 7,9, a pH of 7,7, a pH of 7,8 or a pH of 7,9;
32—the GLP-1 compound is liraglutide, the polycationic compound is protamine, and the liraglutide:protamine molar ratio is 1:0,13-0,15.
33—the GLP-1 compound is liraglutide, the polycationic compound is protamine, and the liraglutide:protamine molar ratio is 1:0,13-0,15, the composition having a pH≧7,7, a pH ≧7,8, a pH between 7,7 and 8,0, a pH between 7,7 and 7,9, a pH of 7,7, a pH of 7,8 or a pH of 7,9;
34—the GLP-1 compound is liraglutide, the polycationic compound is protamine, and the liraglutide:protamine molar ratio is 1:0,11 or 1:>0,11 or is 1:≧0,11, the composition having a pH of 7,7 or a pH≧7,7;
35—the GLP-1 compound is liraglutide, the divalent metal is zinc, the polycationic compound is protamine and the liraglutide:zinc:protamine molar ratio is 1:2,1-2,4:0,13-0,15;
36—the GLP-1 compound is liraglutide, the divalent metal is zinc, the polycationic compound is protamine and the liraglutide:zinc:protamine molar ratio is 1:2,1-2,4:0,13-0,15, the composition having a pH≧7,7, a pH≧7,8, a pH between 7,7 and 8,0, a pH between 7,7 and 7,9, a pH of 7,7, a pH of 7,8 or a pH of 7,9;
37—the GLP-1 compound is liraglutide, the divalent metal is zinc, the polycationic compound is protamine and the liraglutide:zinc:protamine molar ratio is 1:2,2:0,13, 1:2,2:0,14 or is 1:2,2:0,15;
38—the GLP-1 compound is liraglutide, the divalent metal is zinc, the polycationic compound is protamine and the liraglutide:zinc:protamine molar ratio is 1:2,2:0,13, 1:2,2:0,14 or is 1:2,2:0,15, the composition having a pH≧7,7, a pH≧7,8, a pH between 7,7 and 8,0, a pH between 7,7 and 7,9, a pH of 7,7, a pH of 7,8 or a pH of 7,9;
39—any one of embodiments 28 to 38 wherein the composition is in the form of particles or of a suspension of particles, the particles comprising the GLP-1 compound, the divalent metal and the polycationic compound.
40—any one of embodiments 28 to 38 wherein the composition is in the form of particles or of a suspension of particles, the particles comprising a core and a surrounding layer, the core comprising the GLP-1 compound and the divalent metal, and the layer comprising the polycationic compound.
41—the GLP-1 compound is liraglutide, the divalent metal is zinc, and the liraglutide:zinc molar ratio is 1:2,1-2,4, the composition is in the form of particles or of a suspension of particles, the particles comprising a core and a surrounding layer, the core comprising the GLP-1 compound and the divalent metal, the layer comprising the polycationic compound and the core of the particle comprising no polycationic compound.

In one aspect, the invention relates to a method of making the composition of the invention, as defined above.

In one embodiment, the method of the invention comprises one step of mixing of a GLP-1 compound with a divalent metal and one further step of adding a polycationic compound to the GLP-1 compound:metal mixture. The inventors found that this two-step approach allows the formulation of rather high concentrations of the GLP-1 compounds, thus further improving the sustained release of the composition.

In one embodiment, the method of the invention comprises the following steps:

a) mixing of an aqueous solution of the divalent metal, the metal being in the form of a salt, with an aqueous solution of the GLP-1 compound;
b) adding an aqueous solution of the polycationic compound, the polycationic compound being in the form of a salt, and an aqueous buffer solution to the composition obtained from step a), the buffer being preferably added prior to adding the polycationic compound.

In one embodiment, the method also comprises a step c) wherein water is added to the composition obtained from step b), in order to achieve the desired final concentration.

In one embodiment, for the purpose of step a), the GLP-1 compound is dissolved to prepare a stock at an appropriate concentration. The concentration of the GLP-1 stock solution can be in the range of 30-90 mg/mL, having a pH of about 8-9. A stock solution of the divalent metal is prepared with a concentration that can be in the range of 0,5-1,0 M range, having a pH of 5-7 depending of counter ion. In the case of zinc acetate, a 1 M solution has a pH value of about 6,6.

For the purpose of step a), the stock solutions can be mixed directly or pre-diluted before mixing. They are mixed under vigorous agitation or stirring using a magnetic stirrer or propeller in combination with an appropriate mixing container. Another option is to use a static mixer process, as known to persons skilled in the art. During this mixing step, the divalent metal and the GLP-1 compound co-precipitate.

In one embodiment, in the mixing step a), the aqueous solution of GLP-1 compound is added sub-surfacially into the aqueous solution of metal salt, to avoid lump formation, and/or the aqueous solution of GLP-1 compound has an alkaline pH and the aqueous solution of metal salt has an acidic pH. In one embodiment, the pH of the aqueous solution of GLP-1 is about 9,0. In one embodiment, the pH of the aqueous solution of metal salt is about 6,6.

In one embodiment, in the mixing step a), the metal salt solution is added to the GLP-1 solution. In another embodiment, in the mixing step a), the GLP-1 solution is added to the metal salt solution. This latter embodiment avoids the formation of small crystalline particles of zinc hydroxide, especially when the aqueous solution of GLP-1 compound has an alkaline pH and the aqueous solution of metal salt has an acidic pH.

In one embodiment, a buffer solution, such as a solution of TRIS, or TRIS containing a small amount of sodium hydroxide, is added after the mixing of step a) and before the addition of the polycationic compound, to achieve the pH of the final formulation. The use of sodium hydroxide alone, in a liraglutide solution, was not sufficient and the pH of the formulation was found to decrease by standing. The use of sodium phosphate, in a liraglutide solution, may lead to the formation of zinc phosphate crystals in the formulation during standing. For example, by addition of an unadjusted TRIS solution to a final formulation concentration of 25 mM prior to the addition of the polycationic compound, then only minimal or no final pH adjustment is necessary.

In one embodiment, for the purpose of step b), a stock solution of a salt of polycationic compound is prepared. The concentration of the stock solution can be at least 20 mg/mL, depending of the counterion of the chosen polycationic compound. The polycationic compound can be added directly from the stock solution or from a pre-dilution of the stock solution to the aqueous suspension containing the amorphous GLP-1:metal particles.

In one embodiment, the stock solution of the polycationic compound is prepared with NaCl. It has been found that the solubility of the polycationic compound is significantly increased by adding NaCl, at relevant temperatures. This allows the storage and/or use of higher concentrations and lowers the volumes of polycationic compound stock solution needed. This makes much larger volume available for either the GLP-1 compound stock solution or the solution of metal salt. Thereby, it is easier to control the mixing process, involving the aqueous solution of GLP-1 compound and the stock solution of metal salt. As a result of this, the injectability of the final formulation is improved, especially after storage at elevated temperatures. For example, a 30-60 mg/mL protamine sulphate stock solution is achievable at 21° C. when adding sodium chloride corresponding to a concentration of 0,3M, and a 30-80 mg/mL protamine sulphate stock solution is achievable at 21° C. when adding sodium chloride corresponding to a concentration of 0,5M.

The addition of a buffer solution, after the mixing of step a) and before the addition of the polycationic compound, as mentioned above, also allows the adjustment of the pH of the mixture of the above mentioned stock solutions. By carefully selecting the amount of buffer and sodium hydroxide, it is possible to avoid a final adjustment of the pH after addition of the polycationic compound. Thereby, an aseptic process is a lot easier to carry out successfully, by not having to take out an aliquot of the suspension in an aseptical manner, measure the pH and potentially adjust the pH by addition of sodium hydroxide and/or an appropriate acid until the target pH is reached.

In one embodiment, further excipients are added. For example, isotonic agents, such as glycerol or sodium chloride, are used to achieve isoosmolarity with blood serum. The isotonic agent does not need to be added as the last excipient. It can conveniently be added e.g. to the GLP-1 solution before addition to the metal salt solution.

The composition of the invention may also be obtained be selecting an appropriate spray-drying process. The resulting GLP-1-divalent metal-polycationic compound spray-dried composition may be resuspended into an aqueous or non-aqueous vehicle.

Therefore, in another embodiment, the method of the invention comprises the following steps:

a) preparation of a composition comprising the divalent metal and the GLP-1 compound;
b) spray-dry of the composition of step a) to form a powder;
c) re-suspension of the powder obtained from step b) in an aqueous medium or in a non-aqeuous medium;
d) addition of a solution of the salt of the polycationic compound and of a buffer to the suspension obtained from step c).

The following comment apply to the method above as well as the to the methods bellow.

The preparation of step a) is operated like the mixing step a) described above.

For the purpose of step b), the spray-drying method is known to persons skilled in the art. An optimized particle size distribution is achieved.

For the purpose of step c), an aqueous medium can be a suitable iso-osmotic medium known by persons skilled in the art and a non-aqueous medium can be an pharmaceutical acceptable oil known to persons skilled in the art.

The addition of buffer and polycationic compounds at step d) applies when an aqueous final formulation is desired. It is also possible to spray dry the GLP-1:divalent metal particles (without protamine) and have the protamine present in the aqueous resuspension medium.

In another embodiment, when a non-aqueous composition is desired, the addition of a solution of the salt of the polycationic compound and of a buffer of step d) is proceeded during the spray-dry of step b) rather than to the suspension obtained from step c). When added during the spray-dry of step b), the polycationic compound is added as an solution.

In one embodiment, the method is operated under aseptic conditions.

In another embodiment, the method of the invention comprises the following steps:

a) preparation of a composition comprising the divalent metal and the GLP-1 compound;
b) addition of a solution of the salt of the polycationic compound to the composition of step a);
c) spray-dry of the composition of step b) to form a powder;
d) re-suspension of the powder obtained from step b) in an aqueous medium or in a non-aqueous medium;
e) addition of a buffer to the suspension obtained from step d).

In another embodiment, the method comprising the following steps:

a) preparation of a composition comprising the divalent metal and the GLP-1 compound;
b) high-pressure homogenisation and/or ultra-sound treatment of the composition of step a);
c) addition of an aqueous solution of a salt of the polycationic compound and of an aqueous buffer solution to the composition of step b).

When the pharmaceutical composition is a non-aqueous suspension of particles, the particles are preferably obtained by isolation and drying from an aqueous suspension, or made by appropriate spray drying.

When the pharmaceutical composition is an aqueous suspension of particles, the particles are preferably used without isolation or made by appropriate spray drying.

The pharmacokinetic and pharmacodynamic properties of the resulting compositions can be evaluated by animal or clinical studies. The release properties of the compositions can also be evaluated by suitable in vitro release studies.

The chemical and physical stability of the resulting compositions can be evaluated by carrying out standard stability studies, making use of relevant analytical methods appropriate to characterize the GLP-1 compounds or selected excipients; and the composition as a whole.

In another aspect, the invention relates to compositions obtained by the above described methods.

In another aspect, the invention relates pharmaceutical compositions as described above for use as a medicament.

In another aspect, the invention relates pharmaceutical compositions as described above for use as a treatment of metabolic diseases. Non-limiting examples of metabolic diseases include diabetes and obesity.

In another aspect, the invention relates pharmaceutical compositions as described above, for use as a medicament with a frequency of administration below once per day (24 hours) and up to once per week (7 days), twice weekly or below twice weekly, 3 times weekly or below, 4 times weekly or below, 5 times weekly or below, or 6 times weekly or below.

In one embodiment, it is used for the treatment of diabetes by injection administration below once per day (24 hours) and up to once per week (7 days), twice weekly or below twice weekly, 3 times weekly or below, 4 times weekly or below, 5 times weekly or below, or 6 times weekly or below.

In one embodiment, it is used for the treatment of diabetes by injection administration once every 6 days, or once every 5 days, or once every 4 days, or once every 3 days, or once every 2 days, or less frequently than once every 1 day.

In one embodiment, it is used for the treatment of diabetes by injection administration once every 2 to 3 days, once every 3 to 4 days, once every 4 to 5 days, once every 5 to 6 days, once every 6 to 7 days, once every 5 to 7 days, once every 4 to 7 days, once every 3 to 7 days, or once every 2 to 7 days.

In one embodiment, the frequency of administration is below once weekly (7 days), below once monthly, or up to once per month (28, 29, 30 or 31 days), twice monthly or below twice monthly, 3 times monthly or below, 4 times monthly or below, or 5 times monthly or below.

In one embodiment, it is used for a treatment, especially the treatment of diabetes, by injection administration below once weekly (7 days), below once monthly, or up to once per month (28, 29, 30 or 31 days), twice monthly or below twice monthly, 3 times monthly or below, 4 times monthly or below, or 5 times monthly or below.

In one embodiment, it is used for a treatment, especially the treatment of diabetes, by injection administration once every 27-31 days, or once every 22-27 days, or once every 19-21 days, or once every 15-20 days, or once every 12-15 days, or once every 8-12 days, or less frequently than once every week.

In one embodiment, it is used for a treatment, especially the treatment of diabetes, by injection administration once every 27-31 days, or once every 22-27 days, or once every 19-21 days, or once every 15-20 days, or once every 12-15 days, or once every 8-12 days, or less frequently than once every week.

### Examples

(a) A Pharmaceutical Composition of the Invention:

In one embodiment, the pharmaceutical composition of the invention comprises:

| Concentration (mM) | Item | Concentration (mg/mL) |
|---|---|---|
| 10.7 | Liraglutide | 40 mg/ml |
| 23.5 | Zinc Acetate | 4.3 mg/ml |
| 1.5 | Protamine Sulphate | 8.0 mg/ml |
| 25 | Tris | 3.0 mg/mL |
| 72 | Sodium Chloride | 4.2 mg/ml |

pH 7.8
Here the molar ratio of liraglutide:zinc:Protamine is [1:2.2;0.14]

This pharmaceutical composition presents the following beneficial properties:

a sustained release PK-plasma profile, in pig model, with minimal burst release of liraglutide;

a minimum of histamine release, in rat model;

an acceptable low tissue reaction, as shown by histology study in pigs;

a good chemical stability;

an acceptable physical suspension stability;

no liraglutide in supernatant: much less than 0.1% of the total liraglutide concentration;

a minimum zinc and protamine in supernatant;

injectable through a 30G TW needle into either air or subcutaneous tissue at an acceptable dosing rate.

(b) A Method of the Invention:

2,35 ml of a 0,5 M Zn(OAc)₂ solution and 5,0 ml of a 0,3 M NaCl solution are added to a 100 ml beaker containing a magnetic stirrer pin. 25 ml of an aqueous solution of 80 mg/ml liraglutide is added with heavy agitation by a thin cannula beneath the surface. After agitation for 5 minutes, 625 μl of a 2 M TRIS solution (tris(Hydroxymethyl)ami-nomethane) and 100 μl of a 1M NaOH are added. After further agitation for 10 minutes, 8,4 ml of a 50 mg/ml solution of Protamine sulphate in 0,3 M NaCl is added. Water is finally added q.s. (in sufficient quantity) to a formulation volume of 50 ml. The liraglutide:zinc:protamine final molar ratio is 1:2,2:0,15.

The pH of the final formulation is 7,7-7,9.

(c) A Method of the Invention:

2,26 gram of liraglutide (88,4% protein) is dissolved at 23° C. in 22 ml of water and 1,20 ml 3 M NaCl is added and the solution is sterilised by filtration. 1175 μl 1 M Zinc acetate is mixed with 1,0 ml water, sterilised by filtration and added to a preweighed 100 ml sterile borosilicate bottle (BlueCap, Duran®) equipped with a magnet stirrer pin. The liraglutide solution is added under vigorous agitation (about 400 rpm) to the zinc solution beneath the surface at the bottle wall through a long 23G cannula as fast as possible. After further 2 minutes agitation 1250 μl sterilised 1 M TRIS solution is added and followed by 20 ml of sterilised 2% protamine sulphate solution. Sterile water is added up to a formulation weight of 50 gram and the suspension is slowly agitated for 1 hour. The pH is finally adjusted to 7,8 with 2 M NaOH. The bottle is closed and left overnight at 5° C. Next morning the pH is checked at 23° C. and readjusted if necessary to 7,8. The suspension is then filled into injection pens (Penfill® cartridges).

The liraglutide:zinc:protamine final molar ratio is 1:2,2:0,14.

(d) Optimisation of Liraglutide:Zinc Molar Ratios at Various pH-Values:

Liraglutide has been found to precipitate quantitatively from a solution containing Zinc ions. FIG. 1 shows the concentration of free liraglutide (y-axis), in mg of liraglutide per mL of supernatant, for increasing values of zinc:liraglutide molar ratio (x-axis) and various pH. A low concentration of free liraglutide is associated with an efficient incorporation of the compound in the composition, a high stability of the composition and other benefits, including sustained release, injectability and side effects.

In this test, stock solutions of liraglutide were made in 12,5 mM tris buffer and contained 50 mM liraglutide. A zinc acetate stock solution (213,2 mM) was prepared.

For each sample, an appropriate amount of zinc acetate stock solution was added to 0,4 mL of liraglutide stock solution. MilliQ was added to each sample to reach a final volume of 0,5 mL. After whirli-mixing pH was adjusted to 7,8, 8,0, or 8,2. After pH adjustment the samples where whirli-mixed once more. After sedimentation of the particles, supernatant was withdrawn, centrifuged (15.000 G for 15 minutes) and the content of liraglutide in the supernatant was analyzed by UV-spectroscopy.

The results, reported on FIG. 1, show that the concentration of free liraglutide depends on the zinc:liraglutide molar ratio. At pH 7,8, a minimum molar ratio of 2,1:1 is required to obtain no free liraglutide in the supernatant, i.e. 100% of liraglutide is precipitated. At pH 8,0, the minimum molar ratio is 2,1-2,2:1, and at pH 8,2, the minimum molar ratio is >3:1. At pH 7,0, the minimum molar ratio of zinc to obtain 100% liraglutide precipitated is 1,3 (data not reported).

(e) Optimisation of Liraglutide:Protamine Molar Ratios:

With a zinc:liraglutide molar ratio of 2,2:1 as set point, the molar ratio of protamine that optimizes the composition properties was investigated. Protamine increases the sustained release.

In this test, a composition was prepared so as to comprise 10 mM of liraglutide, 22 mM of ZnCl₂ (in the form of Zn2+ in the co-precipitate), which means that the zinc:liraglutide molar ratio is 2,2:1. The pH of the final suspension was 7,8, at 25° C.

Therefore, an amount of liraglutide bulk material corresponding to 1,875 gram of liraglutide (corresponding to 0,5 moles) was dissolved in 30 mL of water. After filtration, 5,5 ml of 0.2 M ZnCl₂ was added to the filtrate containing liraglutide, under vigorous stirring. Afterwards, 1 ml of 1 M Tris buffer, (pH 7.8) was added. Hereafter, the pH was adjusted from pH 7,5 to 7,8 by addition of about 10 μl of 1M NaOH. In each of 8 containers, 10% of the resulting suspension was transferred. Under vigorous stirring, a specific portion of a 18 mM Protamine Chloride solution was added.

The initial container received 0,10 mL of the 18 mM Protamine Chloride solution, followed by 0,2 mL to the second container; ending with addition of 0,8 mL to the final container (number 8). Water was added to each of the eight containers to achieve a total of 5,0 grams of suspension. After 1 hour storage, about 1 mL were withdrawn from each container and centrifuged for 10 minutes at high speed. A portion of clear supernatant was withdrawn from each container and the amount of free Liraglutide and protamine. was measured by a standard liquid chromatographic method.

FIG. 2 shows the concentration of free protamine (y-axis left) and the concentration of free liraglutide (y-axis right) in solution (i.e. the supernatant), in mM, for increasing concentrations of protamine (x-axis), in mM, added to the particles, at pH 7,8 and 25° C. A low concentration of free liraglutide are associated with an efficient incorporation of the compound in the composition, a high stability of the composition and other benefits, including sustained release, injectability and side effects.

The results, reported on FIG. 2, show that the maximal amount of protamine bound to the liraglutide and zinc of the composition, i.e. the maximal amount of protamine that is efficiently incorporated into the composition, is 0,1 mol Protamine per mol liraglutide. Above that ratio, there is protamine present in a free form in the supernatant. The amount of protamine added to the suspension does not influence the solubilisation of free liraglutide.

To ensure a sufficient sustained release of liraglutide, a slight surplus of protamine is convenient. Therefore a ratio of 0,13, 0,14 or 0,15 mol protamine to 1 mol liraglutide is selected.

Liraglutide:zinc:protamine molar ratios of 1:2,1:0,13 or 1:2,2:0,13, 1:2,1:0,14 or 1:2,2:0,14 or 1:2,1:0,15 or 1:2,2:0,15 are selected, with pH 7, 8 or 7,7-7,9.

(f) Optimisation of the pH Value in a Composition with a Molar Liraglutide:Zinc:Protamine Ratio of 1:2,2:0,14:

Here, the amount of protamine remaining free in solution was determined in compositions with different pH-values.

In this test, a composition was prepared so as to comprise 10,7 mM of liraglutide, 23,5 mM of ZnCl₂ (in the form of Zn2+ in the co-precipitate), 1,5 mM protamine sulphate, 40 mM TRIS/HCl, 60 mM NaCl, so that the liraglutide:zinc:protamine molar ratio is 1:2,2:0,14. The pH of the final suspension was tested within the range 7-8, at 23° C., by appropriate adjustment of the final pH. The other details on this experiment is similar to the method in example (e). For each tested pH, both free liraglutide and free protamine were measured in the supernatant by a standard liquid chromatographic method.

FIG. 3 shows the concentration of free protamine (y-axis left) and the concentration of free liraglutide (y-axis right) in solution (i.e. the supernatant), in mM, for increasing pH values. A low concentration of free liraglutide and free protamine are associated with a high stability of the composition and other benefits, including improved sustained release, injectability and side effects.

The results, reported on FIG. 3, show that the minimal concentration of free protamine in the supernatant was found at pH 7,7-7,9. Thus a pH-value of 7,7-7,9 or of 7,8 is selected.

(g) Liraglutide Concentration:

Pharmacokinetic studies (data not reported) have shown that a formulation according to the invention with a liraglutide concentration of 40 mg/ml (10,7 mM) is sufficient for achieving an acceptable level of liraglutide in the bloodstream lasting for 7 days.

(h) Suitable Excipient Stock Solutions for the Manufacturing Process of a Liraglutide-Zinc-Protamine Suspension:

| | |
|---|---|
| 90 mg/ml Liraglutide | 1M Zinc Acetate |
| 1M tris(hydroxymethyl)aminomethane (TRIS | 20 mg/ml Protamine |
| | Sulphate |
| 3M NaCl (Isotonic agent) | 2N NaOH (pH adjustment) |

These solutions are sterile and are applicable for manufacturing of a once weekly liraglutide suspension of the formulation given in example (c).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A composition comprising a GLP-1 compound, a divalent metal and a polycationic compound, wherein the GLP-1:divalent metal molar ratio is 1:>2 and the GLP-1 compound is selected from the group consisting of GLP-1 analogues or GLP-1 derivatives.

2. The composition according to claim 1, wherein the GLP-1:divalent metal molar ratio is 1:≧2,1.

3. The composition according to claim 2, wherein the GLP-1:divalent metal molar ratio is 1:2,1 or is 1:>2,1 or is 1:≧2,2 or is 1:2,2 or is 1:>2,2.

4. The composition according to claim 1, wherein the GLP-1:divalent metal molar ratio is between 1:2,0 and 1:2,4, between 1:2,1 and 1:2,4 or between 1:2,1 to 1:2,3 or between 1:2,2 and 1:2,3.

5. The composition according to claim 1, wherein the GLP-1 compound:polycationic compound molar ratio is 1:>0,10; 1:>0,11; 1:≧0,11; 1:≧0,12; 1:>0,12; 1:0,12-1,0,15; 1:≧0,13; 1:>0,13; 1:0,13-0,15; 1:0,13; 1:0,14-1:0,15; 1:0,14 or 1:0,15.

6. The composition according to claim 1, wherein the GLP-1:divalent metal:polycationic compound molar ratio is 1:>2,0:>0,10 or is 1:≧2,1:≧0,11 or is 1:≧2,1:0,13-0,15 or is 1:2,2:0,13-0,15.

7. The composition according to claim 1, wherein the GLP-1 compound is a GLP-1 analogue with a maximum of 17 amino acid modifications compared to a natural GLP-1 of reference.

8. The composition according to claim 1, wherein the GLP-1 compound is a GLP-1 derivative selected from the group consisting of an amidated, alkylated, acylated, esterified, PEGylated, sialylated and/or a glycosylated parent peptide, the parent peptide being selected from a natural GLP-1 or a GLP-1 analogue.

9. The composition according to claim 1, wherein the divalent metal is selected from zinc (Zn), calcium (Ca), manganese (Mn) or magnesium (Mg).

10. The composition according to claim 1, wherein the polycationic compound is selected from protamine, chitosan, a chitosan derivative, polylysine and polyarginine.

11. The composition according to claim 6, wherein the GLP-1 compound is liraglutide, the divalent metal is zinc, the polycationic compound is protamine, and the GLP-1:divalent metal:polycationic compound molar ratio is 1:>2,0:>0,10 or is 1:≧2,1:≧0,11 or is 1:≧2,1:0,13-0,15 or is 1:2,2:0,13-0,15.

12. The composition according to claim 1, with a pH comprised between 4 and 8,2.

13. The composition according to claim 11, wherein the pH is comprised between 7,2 and 8,2, between 7,4 and 8,2, between 7,4 and 7,9, between 7,6 and 8,0 or between 7,7 and 7,9, or said pH is 7,4; 7,6; 7,7; 7,8; 7,9; 8,0; 8, 1 or 8,2.

14. A method for the preparation of a composition as defined in claim 1, the method comprising one step of mixing of a solution of a GLP-1 compound with a solution of a divalent metal and one further step of adding a solution of a polycationic compound to the GLP-1 compound:metal mixture.

15. The composition according to claim 1 for use as a medicament.
